(19) United States
(12) Patent Application Publication
(10) Pub. No.: US 2013/0046172 A1
(43) Pub. Date: Feb. 21, 2013

(54) METHODS AND SYSTEMS FOR LOCATING A FEEDING TUBE INSIDE OF A PERSON

(71) Applicants: Kathryn A. McKenzie Waitzman, Riverwoods, IL (US); Bradley Frank Slaker, Loretto, MN (US)

(72) Inventors: Kathryn A. McKenzie Waitzman, Riverwoods, IL (US); Bradley Frank Slaker, Loretto, MN (US)

(21) Appl. No.: 13/657,504

(22) Filed: Oct. 22, 2012

Related U.S. Application Data
(63) Continuation-in-part of application No. 12/048,500, filed on Mar. 14, 2008.
(60) Provisional application No. 61/550,397, filed on Oct. 22, 2011.
(60) Provisional application No. 60/906,981, filed on Mar. 14, 2007.

(51) Int. Cl. A61B 6/00

(52) U.S. Cl. 600/424

(57) ABSTRACT

A system for detecting the location of an exit point of an object in a person's body includes a feeding tube, and a light source configured to generate light coupled to a light transmitting element. The light transmitting element transmits the light through an exit point. A light source sensor detects the light and a signal is generated indicating the detection of the light. A method for using the system includes inserting a feeding tube into a patient and generating light that is transmitted to an exit point. Detecting light transmitted from the exit point and generating an indicator signal as a function of the detected light.

### CROSS-REFERENCE TO RELATED APPLICATIONS

This non-provisional application makes reference, and claims priority to U.S. Provisional Application No. 61/550,397, filed on Oct. 22, 2011, by Kathryn Waitzman titled “LOCATION, MAINTENANCE & IDENTIFICATION SYSTEM FOR TEMPORARY OROGASTRIC AND/OR NASOGASTRIC TUBE”; and U.S. Non-Provisional application Ser. No. 12/048,500, filed on Mar. 14, 2008, by Kathryn Waitzman, titled “METHODS AND SYSTEMS FOR LOCATING A FEEDING TUBE INSIDE OF A PATIENT,” which in turn claims priority to U.S. Provisional Application No. 60/906,981 titled “FEEDING TUBE DETECTOR” filed Mar. 14, 2007. U.S. Provisional Application No. 61/550,397, U.S. Non-Provisional application Ser. No. 12/048,500 and U.S. Provisional Application No. 60/906,981 are hereby incorporated by reference herein, in their entireties.

### FIELD OF THE INVENTION

Embodiments of the presently described technology generally relate to techniques to confirm the location of a medical device in a person's body. More particularly, embodiments of the present technology relate to techniques and systems for confirming that the end of a feeding tube has been inserted into person and for locating the same therein.

### BACKGROUND OF THE INVENTION

Feeding tube intubation is a process involving placement of a soft plastic tube into a patient's gastrointestinal system ending in the stomach or jejunum, otherwise referred to as the small intestine. The gastric or intestinal tube,, such as a nasogastric (NG), orogastric (OG) tube, surgically implanted gastric tube, a foley catheter or a sump drainage device, is inserted into a person. The tube may be inserted through a patient's nose or mouth and travels past the pharynx, down the esophagus and into a patient's stomach or beyond to the small intestine. Intubation is a common medical practice that may assist in the treatment and diagnosis of patients. For example, the intubation of a gastric feeding tube may aid a patient in recovery from surgery or trauma by administering life sustaining nutrition or medications where necessary. Persons who need gastric or intestinal feeding tubes include but are not exclusive to pre-mature neonates, comatose patients, patients requiring mechanical ventilation, chronically ill children, patients requiring face or neck surgeries, cancer patients, and/or post-op surgical nutrition. The feeding tubes are considered temporary, non-surgical, and are intended to remain in use for short-term or long-term therapies until a trained physician deems a change medically necessary.

Gastric feeding tube placement is routinely practiced in both medical facilities and in the treatment of in-home-care patients. Intestinal feeding tube placement frequently requires the use of more specialized placement techniques and the placement position is more difficult to confirm. As such, intestinal feeding tube placement is predominantly practiced only in medical facilities.

Feeding tubes are routinely placed in patients using a blind technique, with the operator not knowing the true location of the end of the tube after placement, Accordingly, the end of the feeding tube is commonly misplaced inside of the patient, which may lead to serious problems. For example, where a feeding tube intended for placement in the stomach is not placed deep enough, fluids administered through the feeding tube may seep into the lungs causing problems for the patient, Alternatively, where such a feeding tube is placed too deep, the fluids may be absorbed directly into the intestine, which may not have the appropriate enzymes fox processing the fluids, which may also lead to problems. Complications that may result from the improper administration of fluids through an improperly placed feeding tube may include, but are not limited to, pneumothorax, perforated bowel, pneumonia, intestinal distention, aspiration, peritonitis, or placement of the tube into the brain, for example. See, Ellet, Maahs, and Forsee, Prevalence of Feeding Tube Placement Errors and Associated Risk Factors in children, American Journal Maternal Child Nursing, 23:234-39, published 1998; Ellet, What is Known About Methods Of Correctly Placing Gastric Tubes in Adults and Children, Gastroenterology Nursing, 27 (6); 253-59, published 2004; Ellet, What is the Prevalence of Feeding Tube Placement Errors and What are the Associated Risk Factors?, The Online Journal of Knowledge Synthesis for Nursing, 4, document 5, published 1997.

The misplacement of feeding tubes in patients happens frequently when blind insertion techniques are used. Research has suggested that blind placement methods of feeding tubes may have a mal-position rate in pediatric and adult patients of up to 40%. See, Metheny and Tiller, Assessing Placement of Feeding Tubes, American Journal of Nursing, 101:36-41, published 2001; Metheny and Meert, Monitoring Feeding Tube Placement, Nutrition in Clinical Practice, Vol. 19, no, 5, pp. 487-95, published 2004; Huffman, Karczk, O'Brien, Pieper and Bayne, Methods to Confirm Feeding Tube Placement: Application of Research in Practice, Pediatric Nursing, 30:10-13, published 2004; Westhaus, Methods to Test Feeding Tube Placement in Children, The American Journal of Maternal/Child Nursing, 29:282-87, published 2004; Ellet, How Accurate is Enteral Tube Placement in Children?, MNRS Connection, 14 (1), 14, published 1998. Accordingly, it is often necessary to confirm the location of the feeding tube prior to the administration of any medication or nutrition to avoid problems caused by feeding tube misplacement.

Conventional, methods for locating the position of a feeding tube or tubes inside a patient include the use of air insufflation, gastric pH detection methods, gastric enzyme detectors and CO2 detectors. There are problems, however, with the accuracy and reliability of these methods. See, Gharpure, Meert, Sarnaik and Metheny, Indicators of Postpyloric Feeding Tube Placement in Children, Critical Care Medicine, 28:2962-66, published 2000; Metheny, Stewart, Smith, Yan, Diebold and Clouse, pH and Concentration of Bilirubin in Feeding Tube Aspirates as Predictors of Tube Placement, Nursing Research 48, 189-97, published 1999; Araujo-Preza, Melhado, Gutierrez, Maniatis and Castellano, Use of Capnometry to Verify Feeding Tube Placement, Critical Care Medicine, 30:2255-2259, published 2002. For example, air insufflation techniques require a user to confirm the location of a tube by listening for a sound of air passing through a feeding tube inside the patient using a stethoscope. Internal noises may lead to a false confirmation of proper placement, for example. Furthermore, feedings and medications may affect the levels of ph, enzyme and CO2 in a patient, thereby affection the ability of gastric pH, gastric enzymes, and CO2 detectors to produce accurate and reliable results.

Moreover, conventional methods typically require the implementation of equipment that is only available in a hospital or clinical setting and are thus unavailable for use with in-home-care patients. Presently, only air insufflation, the least accurate of the methods, is available to confirm proper placement of feeding tubes for in-home-care patients.

In June 2005, the American Association of Critical-Care Nurses (AACN) issued a practice alert. The alert recommended using an X-ray to visualize a new, blindly inserted gastric tube to ensure that the tube has been properly placed and is in the desired position of the stomach or small intestine before initiating the administration of formula or medications via the tube. See, American Association of Critical Care Nurses, Practice Alert—Verification of Feeding Tube Placement, May 2005. Though more accurate than the conventional methods described above, the use of such techniques typically requires at least 5 X-ray scans to confirm the location of the tube, for each time as intestinal feeding tube is inserted blindly at a patients hospital bedside. It is not uncommon for children and neurologically compromised patients to personally remove/extubate the OG or NG tubes more than one time daily which would require additional X-rays for each new tube placement. Such persistent exposure to X-rays throughout a patients treatment gives rise to serious concern, as the high levels of radiation may have harmful effects on the patient. This concern is especially great where the patient is a child. An additional disadvantage for using X-ray techniques to confirm feeding tube placement is that the equipment necessary to perform the techniques is typically only available in hospital environments and thus of no help to in-home-care patients.

Recently, the use of electromagnetic tube placement devices has provided a means to increase the accuracy of feeding tube placement without the need for X-ray exposure to patients. An example of such a device is the CORTRAK™ system produced by Cardinal Health. (A description of the product is available on the Cardinal Health website, at www.viasyshealthcare.com/prod_serv/prodDetail.aspx?config=ps yrodDtl&prodID=276, as of Mar. 11, 2008). The electromagnetic systems involve the placement of an electromagnetic transmitter inside of the feeding tube. As the tube is inserted into the patient, an electromagnetic tracking device tracks the position of the feeding tube, and displays the location on a display unit. Accordingly, operators may respond immediately where a tube placement does not follow the expected path. Because these techniques are only available in medical facilities, they are not helpful when needed for in-home-care.

Once the feeding tube has been inserted into the patient using the aforementioned electromagnetic tracking techniques, the transmitter device must be removed before feedings or medications may be administered through the tube. After the transmitter has been removed, however, it may not be reinserted without the removal of the feeding tube. Accordingly, once the transmitter has been removed, the position of a feeding tube inside the patient may not be checked. This shortcoming of the electromagnetic system is significant, as patient movement, periodic adjustment of the equipment, peristalsis and other internal functions all contribute to constant shifting and relocation of the feeding tube. Thus, it is necessary to periodically confirm the position of a feeding tube, even after it has been inserted. Without the transmitter located in the tube, the electromagnetic tracking techniques cannot confirm the position after insertion without the use of X-rays.

Thus, the concerns with the present feeding tube placement practices and techniques include several problems relating to accuracy, safety and ease of use for in-homecare patients. Thus a need exists for a method and/or system for detecting, and periodically re-checking, the location of a placed feeding tube in a patient's stomach or small intestine that has the accuracy of x-ray detection without the radiation exposure.

### SUMMARY OF THE INVENTION

One or more embodiments of the presently described technology provide a system for detecting the location of a feeding tube inside of a patient's body. In one or more embodiments the system includes a feeding tube, and a light source configured, to generate light coupled to a light transmitting element. The light transmitting element transmits the light through an exit point. A light source sensor detects the light and a signal is generated indicating the detection of the light.

In one or more embodiments, the system may provide a feeding tube embedded with a light source sensor. The light source sensor may be a passive infrared sensor or a fiber-optic filament or filaments. The light source sensor may be connected to a receiver. The system may provide a light source that generates non-radiographic radiation each as light. For example, the light source may generate visible or infrared light. The light source may generate light over the body of a patient in which a feeding tube having a light source sensor has been inserted. At or around the time the light source sensor detects light from the non-radiographic light source, the receiver may generate a signal based on the detection of the light. The receiver may generate a signal based on the intensity of the light detected. The signal may be audible or visual, or the signal may change based on the intensity of light detected by the sensor. For example, the signal may be a tone that increases in volume or pitch as the intensity of light detected by the sensor increases.

In one or more embodiments, a technique for locating the end of a feeding tube in a person's body may include inserting a feeding tube into a patient and generating light that is transmitted to an exit point. The technique may also include detecting light transmitted from the exit point and generating an indicator signal as a function of the detected light.

In one or more embodiments the technique comprises inserting a feeding tube including a light source sensor located at the distal end of the tube into a person. In one or more embodiments, the light source sensor may be connected to a receiver, and the feeding tube may be inserted into the person. A non-radiographic radiation such as light may be generated (e.g., illuminated) over the person. The light source sensor may detect the light, and the receiver may generate a signal based on the detection of light. In one or store embodiments, the receiver may generate a signal based on the intensity of light detected at the light source sensor. The non-radiographic light source may be moved externally over the person to the point where the indicator signal generated by the receiver exceeds a predetermined intensity threshold (e.g., where the predetermined intensity threshold is sufficient to confirm the presence of the feeding tube).

A system for locating the end of a feeding tube in a person's body using a proximity sensor and a proximity sensor target may include a feeding tube having a proximity sensor target. The proximity sensor target may be, for example, a metal band or metal object of soma sort. The system may provide a detector including a proximity sensor. For example, the proximity sensor may be a metal detector. The detector may be moved externally over a person's body such that, when the sensor detects the proximity sensor target of the feeding tube, the proximity sensor may produce an indication signal.

### BRIEF DESCRIPTION OF THE DRAWINGS

FIG. 1 illustrates a perspective view of an example of a feeding tube.

FIG. 2 illustrates a top view of an example of a detector.

FIG. 3 illustrates a side view of the detector of FIG. 2.

FIG. 4 illustrates a bottom view of the detector of FIG. 2.

FIG. 5 illustrates a perspective view of an example of a location, maintenance, and identification system.

FIGS. 6A-D illustrate cross-section views of examples of feeding tubes.

FIG. 7 illustrates a perspective view of an example of a light source.

FIG. 8 illustrates a perspective view of an example of a sensor.

FIG. 9 illustrates a perspective view of a receiver.

FIG. 10 illustrates a perspective view of the location, maintenance, and identification system and a receiver.

FIG. 11 illustrates a perspective view of an example of a location, maintenance, and identification system.

FIG. 12 illustrates a top view of the location, maintenance, and identification system of FIG. 9.

FIG. 13 illustrates a cross section of a feeding tube of the system of FIG. 9.

FIG. 14 illustrates a logical block diagram of an example of the location, maintenance, and identification system.

Before the embodiments of the technology are explained in detail, it is to be understood that the technology is not limited in its application to the details of construction and the arrangement of the components set forth in the following description or illustrated in the drawings. The technology is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION

The systems and techniques disclosed herein, in some embodiments, may include a device for determining the location of a feeding tube inside a person. The systems may locate the end of a feeding tube inside a person while limiting the person's exposure to unnecessary radiation and/or light energy, such infrared light. In one or more embodiments, the system may include a feeding tube, which may be inserted into the person. The feeding tube may include a light transmitting element that may transmit light from a light source at a proximal end of the feeding tube to one or more exit points at a distal end of the feeding tube reside the person where the light may be emitted. A sensor may be included that may detect the light being emitted. The sensor may be placed or moved about a location external to the person. At or around the time the sensor detects the emitted light a signal may be generated indicating to a user that the light has been detected. Other embodiments are also described, where, for example the sensor may be internal to the person and the light source may be external. However, this latter embodiment may suffer from being prone to exposing a user and/or the person to additional light energy. Where the light source is infrared, for example, a user may be prone to damaging their eyesight or another's eyesight.

The system may be advantageous because the feeding tube may be left inside the person for an extended period of time and the location of the feeding tube may be checked multiple times during that period. As such, the system may be used to determine the location of a feeding tube one or more times while also limiting a person's exposure to unnecessary radiation and/or light energy, such as infrared light. For example, a system where the light is being transmitted through an exit point or exit points inside of the person (as opposed to being outside with an internal sensor) may reduce or eliminate a user's exposure to unnecessary radiation and/or infrared light and may prevent the user from inadvertent harm to the user's eyes.

Proximity sensors are sensors that are able to detect the presence of nearby objects without physical contact. A proximity sensor emits an electromagnetic or electrostatic field, or a beam of electromagnetic radiation such as infrared, for example. The proximity sensor looks for changes in the field or return signal. The object being sensed is often referred to as a proximity sensor target. Different proximity sensor targets demand different sensors. For example, a capacitive or photoelectric sensor might be suitable for a plastic target, and an inductive proximity sensor may be suitable for a metal target. Proximity sensors have a maximal distance at which they may be detected. The range in which the sensor may be detected is called the “normal range.” A sensor may have the ability to adjust the nominal range, or provide a way to report graduated detection distance. Proximity sensors have a high reliability and a long functional life because of the absence of mechanical parts and the lack of physical contact between the sensor and the sensed object.

A capacitive proximity sensor is a variety of proximity sensor that detects the location of an object between two capacitor plates. When the sensed object moves within the nominal range of the sensor, the dielectric constant between two plates changes, and the position of the object may thus be located. One type of capacitive proximity sensor is a metal detector. One embodiment of the present technology employs the use of a metal detector capacitive proximity sensor to detect the location of a feeding tube inside of a person.

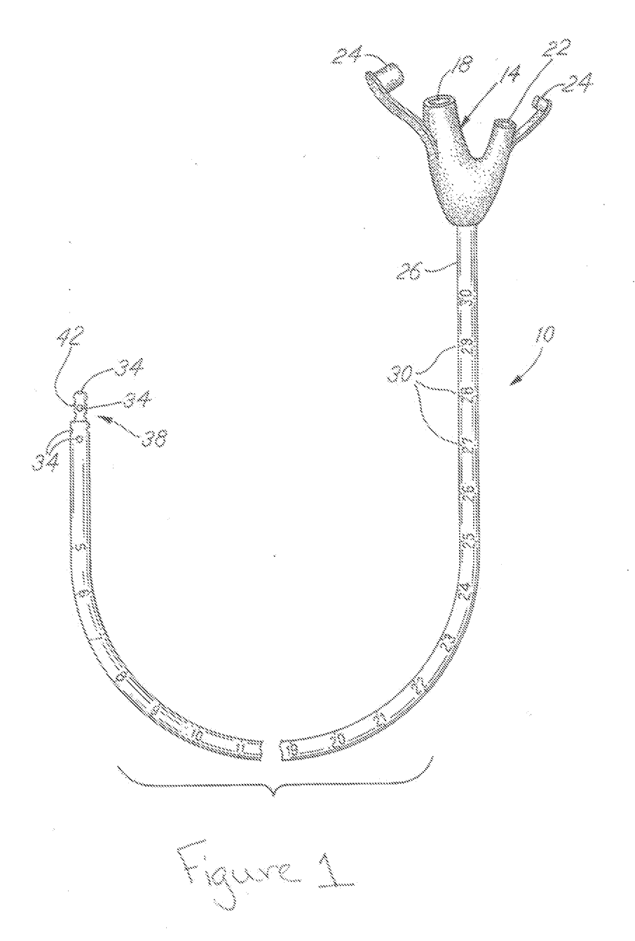

FIG. 1 illustrates a perspective view of a feeding tube system 10 according to an embodiment of the present technology. The feeding tube system 10 has a “Y” port 14 with dual administration ports 18 and 22. The dual ports 18 and 22 may be used simultaneously to administer feedings and medications. For example, the port 18 may be used to receive gastric feeding and the port 22 may be used for medication, flushing, or as a racking port, or vice versa. The “Y” port 14 includes caps 24 connected thereto that may be used to close the administration ports 18 and 22. The “Y” pert 14 is connected to a main tube 26, or a feeding tube.

For identification purposes, the tube 26 may also be referred to as a “feeding tube,” an “oral gastric tube (or OG-tube),” a “nasogastric tube (or NG-tube),” or an “intestinal tube,” depending on the location of placement of the tube inside of a person for treatment. The tube 26 may be comprised of a variety of materials, for example, polyurethane. In one or more embodiments, the tube 26 may include location or measurement markings 30 along the length of the tube 26 from the “Y” port 14 to a distal end 38. The markings 30 may be used as a guide to determine the location of, or the amount of the tube exposed. For example, the markings may be numbered and consistently spaced apart to measure length in, by way of example only, centimeters. In one or more embodiments, an operator may insert the feeding tube 26 to a predetermined depth using the markings 30 as a guide before attempting to confirm location of the end of the tube 26.

The tube 26 may include a radiographic pigment indicator line. The radiographic pigment indicator line allows the tube 26 to appear on an X-ray, should confirmation by X-ray be necessary. Holes 34 may be situated at the distal end 38 of the tube 26 for fluid administration into the person. In one or more embodiments, the tube 26 may also comprise a proximity sensor target 42 impregnated into the distal end 38 of the tube 26. In one or more embodiments, the proximity sensor target 42 may be a metal band or a metal object of some kind, for example. In operation, where the target 42 is a metal, it is preferred that it be comprised of a non-ferrous metal that is not otherwise located in the person's body. For example, where a person has an implant made of titanium, it is preferred that the metal band 42 be comprised of a non-ferrous metal that is not titanium. By way of example only, the metal sensor may be comprised of sine or silver.

In one or more embodiments, the tube 26 may be for single use and be non-sterile. In other embodiments, however, the tube 26 may be reusable. The tube 26 may range in size from 3.5 French (or approximately having a circumference of 3.5 millimeters) to 12 French (approximately 12 millimeters in circumference), for example, and may vary in length. For example, the tube may be, by way of example only, 36 to 42 inches in length.

In operation, the distal end 38 of the feeding tube 10 may be inserted into the stomach or small intestine of a person by inserting the tube 26 through a person's nose or month, and down the person's esophagus such that the distal end 38 locates in the person's stomach, or through the stomach and into the small intestine. Once situated, food and medication may then be fed into the ports 18 and 22, through the tube 26, and into the person through the holes 34 at the distal end 38.

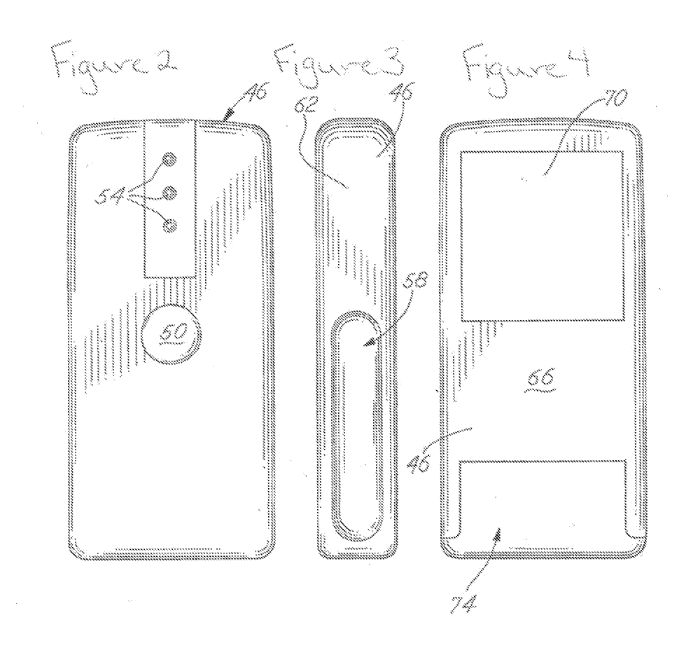

FIG. 2 illustrates a top view of a sensor or detector 46 according to an embodiment of the present technology. The detector 46 is used to determine the position of the proximity sensor target 42 at the distal end 38 of the feeding tube 10. The detector 46 is generally rectangular in shape and sized to be hand-held. By way of example only, the detector 46 may have a housing made of bard plastic.

In one or more embodiments, the detector 46 internally carries a proximity sensor. In one or more embodiments where the proximity sensor target is metal, such as is described above, the proximity sensor may be a metal detector. Externally, the detector includes an on/off button or switch 50 and indicator lights 54. In one or more embodiments the detector may include a speaker instead of, or in addition to, the indicator lights 54. In one or more embodiments, the detector 46 is designed to be operable with either a right or a left hand. For example, a user holding the detector 46 with a left hand only may be able to operate the on/off button, or any other functions, as would a user holding the detector 46 with the right hand only. The metal detector or proximity sensor does not generate X-rays.

In one or more embodiments, the proximity sensor may be capable of scanning for sensor targets (e.g., metal where the proximity sensor is a metal detector) at least at two various depths. For example, the proximity sensor may have a regular depth of scanning and a deeper depth of scanning for obese persons. The detector 46 may have buttons or switches that allow the operator to set the depth of the scan to regular or deep. Alternatively, the defector 46 may be used to scan at any number of different depths.

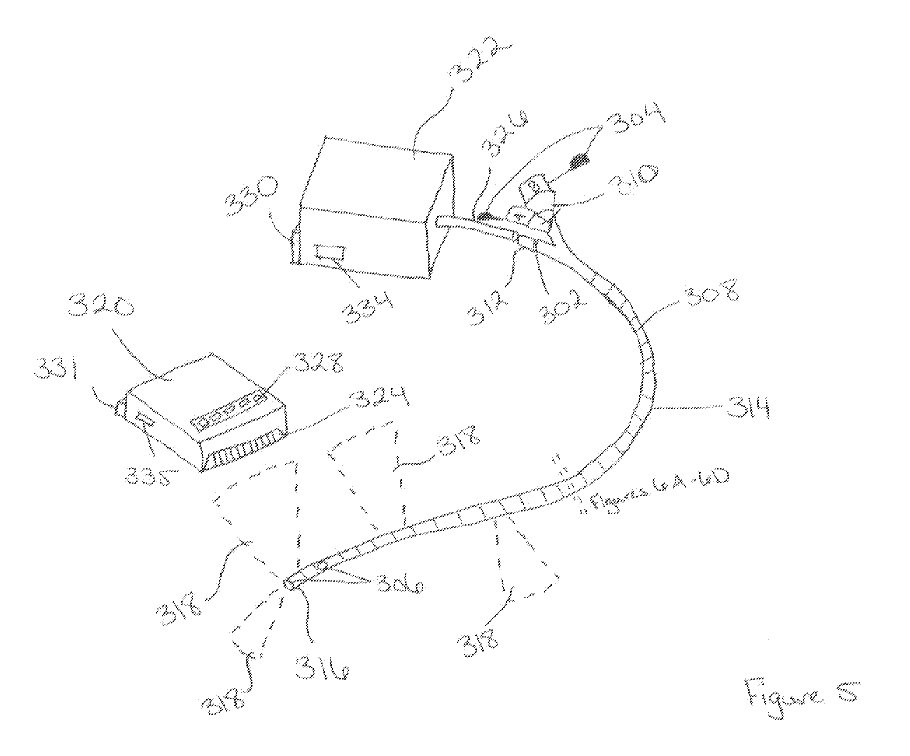

FIG. 3 illustrates a side view of the detector 46 of FIG. 2. The detector 46 includes finger grips 58 along sidewalls 62 thereof. The finger grips may provide for easier gripping of the detector 46 by an operator and may be made of rubber or plastic.

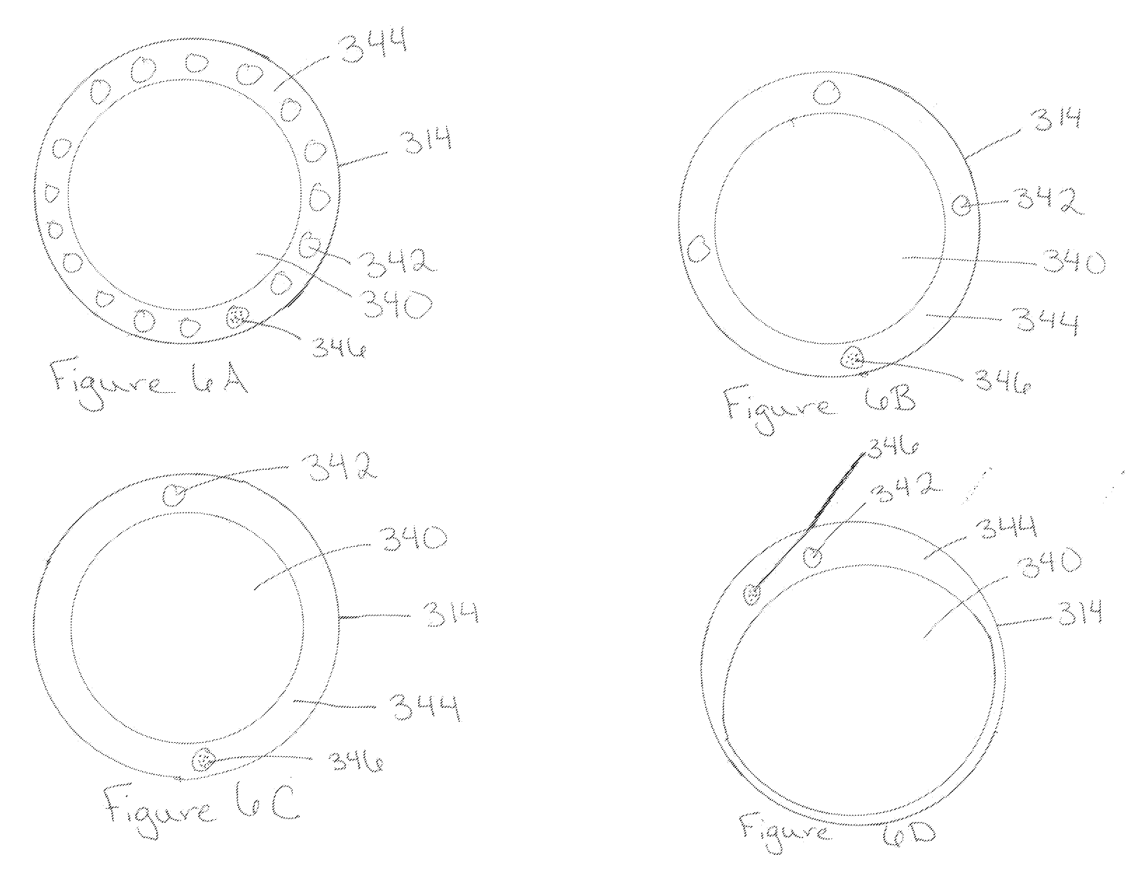

FIG. 4 illustrates a bottom view of the detector 46 of FIG. 2. In one or more embodiments, the detector 46 has a back side 66 that includes a section 70 tor displaying information, for example, use, cleaning and warning instructions. In one or more embodiments, the back side 66 also includes a compartment 74 that may be opened and closed to receive a battery. By way of example only, the detector 46 may operate on a 9-volt battery. In one or more embodiments, the detector 46 may operate with a lithium ion battery. In one or more embodiments, the detector 46 may operate on alternating current or an alternative power source.

Returning to FIG. 2, in operation, an operator presses the button 50 to turn on the defector 46. The operator then moves the backside 66 (FIG. 4) of the defector 46 to the area of the person where the feeding tube 26 is assumed to be situated. For example, where the feeding tube is intended to be located in the person's stomach, the operator moves the back side 66 of the detector 46 externally over the stomach area of a person. The indicator lights 54 may be of one or more colors or indication signal when the detector 46 does not detect the proximity sensor target 42 of the feeding tube 10. For example, the indicator lights 54 may be red when the metal detector 46 does not detect the sensor target 42 of the tube 10 in the person's stomach. When the proximity sensor of the detector 46 detects the sensor target 42 of the feeding tube 10, the lights turn to a different color or indicator signal to indicate that the detector has detected the position of the sensor target 42, and thus the distal end 38 of the feeding tube 10. For example, the indicator lights 54 may turn green to indicate that the sensor target 42 is detected. In another embodiment, the indicator lights 54 may be unilluminated when the detector 46 does not detect the target 42, and illuminated when the detector 46 detects the target 42. In an alternative embodiment, the detector 40 may emit an audible sound to confirm detection in addition to, or instead of, using the indicator lights 54. When the detector 46 has indicated that it has detected the sensor target 42, the operator will be able to determine whether the distal end 38 of the feeding tube is correctly positioned in the stomach.

The embodiments depicted in FIGS. 1-4 and described supra involve use of a sensor target 42, such as a metal band, and a proximity sensor, such as a metal detector, as a way of detecting the position of a feeding tube inside of a person. Other embodiments of the present technology provide alternative techniques for locating the position of a feeding tube inside of a person without exposing the person to radiation. For example, one or more embodiments provide for a sensor on the feeding tube that detects the presence of a source external to the body. Alternatively, one or more embodiments provide for a light transmitting element coupled to a feeding tube and a sensor external to the person detecting the transmitted light. For example, FIGS. 5-14 depict embodiments of the present technology that use a light source sensor and a light source to determine the location of the tube.

A passive infrared sensor (PIR sensor) is an electronic device that measures infrared light radiating from objects within the field of view. PIR sensors are often used in the construction of motion detectors. All objects emit an energy called “black body radiation.” This black body energy is invisible to the human eye, but may be detected by electronic devices. The term “passive” means that the sensor does not emit energy; instead, the sensor merely receives the energy. For example, a PIR sensor detects motion when one infrared source having one temperature, such as a human, passes in front of an infrared source having another temperature, such as a wall. The PIR sensor detects the change in energy between the sensor and the wall and transmits a signal that an object has been detected. One or more embodiments of the present technology employ related systems and techniques to detect the presence of a feeding tube inside of a person.

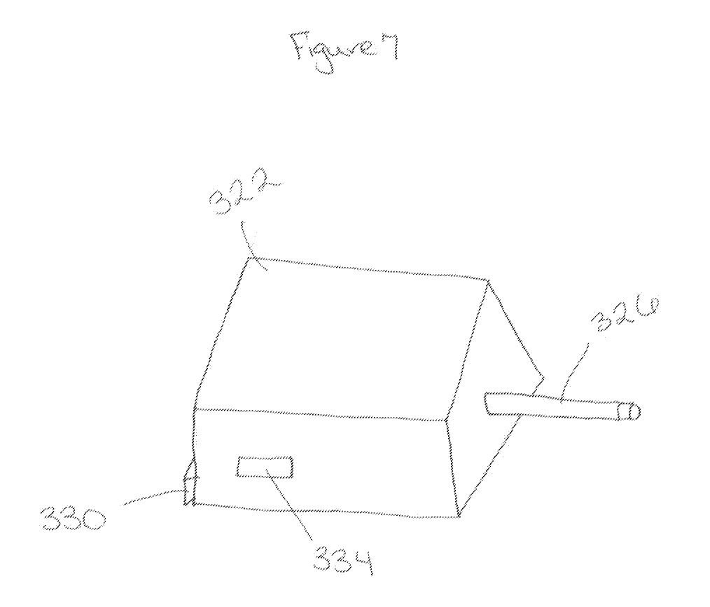

FIG. 5 illustrates a perspective view or an example of a location, maintenance, and identification system. The location, maintenance, and identification system may include a feeding tube 314 having a light transmitting element 346 (see FIGS. 6A-6D) and the system may also include a light source 322 and a light source sensor 320.

The feeding tube 314 may be configured for administering feedings, medications, and/or other deliverables directly to an internal location such as a stomach or small intestine, for example, of a person or animal. The feeding tube 314 may be equipped with a feature allowing the system to provide location identification such that the feedings and other deliverables may be delivered to the desired location within the person or animal.

The feeding tube 314 may extend from a proximal end 302 located outside a person to a distal end 316 at a desired location inside the person and may be configured for delivery of the mentioned feedings and other deliverables. In some respects, the feeding tube 314 may be similar to the feeding tube 26 of FIG. 1. The feeding tube 314 may be attached to a “Y” port 310 at or near the proximal end 302 of the feeding tube 314. The feeding tube 314 may include one or more exit points 313, through which light may be transmitted, at or near the distal end 316 of the feeding tube 314.

The tube 314 may extend from the “Y” port 310, to the distal end 316. The tube 314 may be generally cylindrical in cross-section and may be generally elongate and adapted to extend through a person's mouth or nose and downward through the esophagus, for example, and into the stomach or small intestine. The tube 314 may be at least partially made of plastic or other polymeric material including pebax®. Still other plastic or non-plastic material may also be used. The tube 314 may be a single or multi-durometer tube 314. The tube 314 may be a multi-lumen tube, such as depicted in FIGS. 6A-6D. In some embodiments, one or more of the lumens 342 may be within a wall of the tube, the lumens 340 or 342 may be non-contiguous, and may vary in diameter, and/or length of the lumens 340 or 342.

The tube 314 may include measurement markings 308 along the external longitudinal length thereof. The measurement markings 308 may demarcate the length of the tube 314 from the proximal end 302 to the distal end 316. The length may be demarcated using standard or metric demarcations, such as inches, centimeters, or other standard or metric unit measurement demarcations. The tube 314 may include a radio-opaque indicator line extending along its length or transversely across the tube 314 that will appear on an image generated during an X-ray, Accordingly, when viewed on X-ray a user may be able to view the path of the tube extending into the person, or a user may be able to view the location of an indicator line. The tube 314 may include one or more holes 306 at the distal end 316 thereof for administration of feedings, medications or other substances into or out of the gastrointestinal system of the person. Additionally, the tube 314 may include color impregnated lines to demarcate an expected depth placement based on a person is size and/or weight. For example, alternatively or in addition to the measurement markings 308, colored markings may extend transversely across the tube indicating a suitable depth for a particular person's size and/or weight.

The “Y” port 310 may include dual administration ports A and B. The dual administration ports A and B may be used to drain or administer feedings, medications, or other treatments. For example, port A may be used to receive gastric feedings and port B may be used to administer medication, or as a back up port, or vice versa. The “Y” port 310 may include one or more caps 304 that may be used to close the administration ports A and B. The “Y” port 310 may be connected to the main tube 314.

The tube 314 may be configured to include a light transmitting element 346 for transmitting light 360 (see FIG. 10) from a proximal end 302 of the tube 314 along the length of the tube 314 through an exit point 318 at or near the distal end 316 of the tube 314. The light transmitting element 346 may include a fiber optic-type device, for example, that may receive light 360 (see FIG. 10) at one end and transmit the light 360 to the opposite end. In one or more embodiments, the light transmitting element 346 may be optically connected to a light source 322 at a proximal end 302 of the tube 314 and may extend to one or more exit points 318 at a distal end 316 of the tube. In one or more embodiments, the light transmitting element 346 may include at least one fiber optic filament embedded an at least one lumen 342 of the feeding tube 314. In another embodiment, the light transmitting element 346 may include optically transmittable materials, such as polymethylmethacrylate. The light transmitting element 346 may include a stylet configured to transmit light 360 that may be inserted into or removed from a lumen 340 or 342 of the feeding tube 314. In one or more embodiments, the light transmitting element 346 may include one or more fiber optic filaments that are beveled at an angle to direct the light 360 through one or more exit points 318 which may not be directly at the distal end 316 of the feeding tube. In other embodiments, the filament may have a square end and the filament may be directed generally perpendicular to the tube surface near the distal end 316. Still other geometries and treatments of the filament may be provided to direct the light 360 generally transverse to the longitudinal axis of the tube 314.

The light transmitting element 346 may include more than one fiber optic filaments. Each fiber optic filament may be configured to transmit a different wavelength, frequency or magnitude of oscillations of light 360. In one or more embodiments, at least one of the fiber optic filaments may function to send back to the proximal end 302 of the tube 314 the reflected light from within the person. In one or more embodiments, a reflector may be placed at or near the distal end 316 of the tube 314.

The tube 314 may include multiple exits points 318 through which light may be emitted. In one or more embodiments the exit point 318 may be the location at which the light transmitting element 346 ends. The exit point 318 may be the location along the length of the tube 314 where a fiber optic filament ends. The exit point 314 may be the end point of the light transmitting element 346 where the light transmitting element 346 transmits light 360 through the material of the tube 314.

FIGS. 6A-6D show cross sectional views of examples of tubes 314 including light transmitting element 346, such as the tube 314 depicted in FIG. 5. The tube 314 may include at least one lumen 340 that may be used to administer feedings or medical treatments, among others, and an outer wall 344. The feeding tube 314 may include one or more lumens 342 embedded in the outer wall 344. In one or more embodiments, the tube 314 may include a light transmitting element 346. The light transmitting element 346 may be one or more fiber optic filaments embedded in one or more lumens 342 in the outer wall 344 or the tube 314. In cases where multiple filaments are provided for transmitting differing wavelengths of light, the differing wavelengths of light may be transmitted through differing wall lumens 342.

FIG. 6A illustrates a cross-section of the tube 314 including a feeding lumen 340 for administration into or out of the gastrointestinal region. Additionally, the tube includes a light transmitting element 346 embedded in one or more lumens 342 embedded in the outer wall 344 radially spaced around a circumference of the tube 314. FIG. 6B illustrates a cross-section of the tube 314 including a feeding lumen 340 and an outer wall 344, which includes multiple embedded lumens 342. A light transmitting element 346 embedded in a lames 342 of the outer wall 344 of the tube 314, in one or more embodiments the light transmitting element 346 maybe a single or a plurality of fiber optic filaments. FIG. 6C illustrates a cross-section of a tube 314 that includes a feeding lumen 340, and two lumens 342, including a light transmitting element 346, embedded in the outer wall 344. FIG. 6D illustrates a non-concentric feeding lumen 340, with regard to the circumference of the tube 314. The non-concentric feeding lumen 340 creates a non-uniform thickness of the outer wall 344. The lumens 342 are embedded in the portion of the outer wall 344 with the greater thickness. The lumens 342 may include a light transmitting element 346. In one or more embodiments, the light transmitting element 346 may be a stylet that may be inserted into a lumen 340 or 342 of the tube 314.

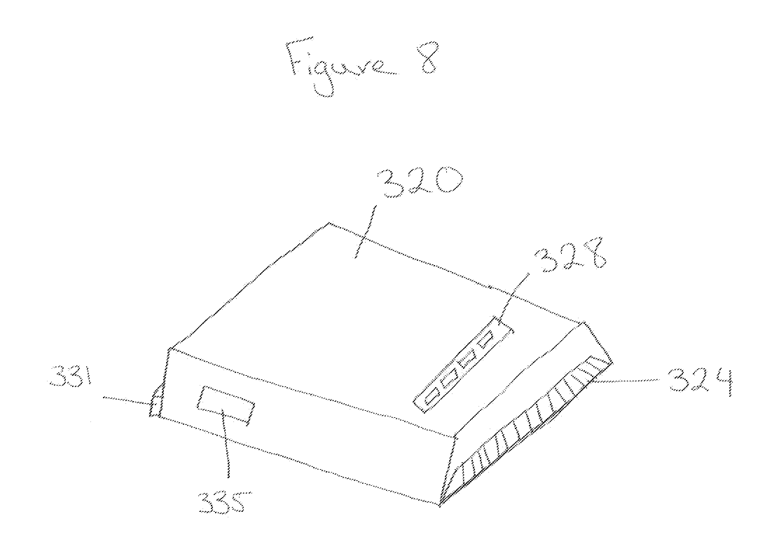

FIG. 7 illustrates a perspective view of the light source 322. The light source 322 may include a power source 330, and a power switch 334. In one or mere embodiments, the light source 322 may couple to the light transmitting element 346 by a pigtail or extension 326 and a joint 312 (as shown in FIG. 5). The light source 322 may generate light 360 when powered on. The light source 322 may be powered on or off by depressing a button or switch 334. The light source 322 may include a power source 330. The power source 330 may be a battery, a power cord plugged into an electrical receptacle, or other source or electrical power. The light source 322 may oscillate the intensity of the light 360 it emits and may emit a variety of wave forms including sine, square, triangular, or a combination thereof, among others. in one or more embodiments the frequency of the light 360 generated by the light source 322 is in the infrared range, or other frequency which penetrates tissue but does not harm the person. Frequencies in or near the infrared range are advantageous because of the ability of these frequencies to penetrate tissue without causing harm to the person.

The light source 322 may be coupled to the light transmitting element 346 in a manner, which may allow the light source 322 to be uncoupled when the system is not being used. When the light source 322 is coupled to the light transmitting element 346, light 340 may be transmitted through at least one exit point 318, such as at or near the distal end 316 of the feeding tube 314. The light source 322 may be coupled to the light transmitting element 346 by a pigtail or extension segment 326 and a “joint” 312 (see FIG. 5). A ferrule connector may be used to couple the light source 322 and the light transmitting element 346 inside the feeding tube 314.

The light source 322 may be coupled to the light source sensor 320 in a manner that does not interfere with the use of the light source 322 or the light source sensor 320. For example, the light source 322 and light source sensor 320 may be tethered by a cord or cable to each other in a fashion that allows the tether between the light source 322 and sensor 320 to be extended during use. In one or more embodiments, the light source 322 and the light source sensor 320 may be wirelessly coupled. In one or more embodiments where the light source 322 and the sensor 320 are coupled, the detection of light 360 by the sensor 320 may cause the light source 322 to be powered off to avoid having the light source 322 generate more light 360 than is necessary to locate the emit point 318 at or near the distal end 313 of the feeding tube 314. In still other embodiments, a delay is included after the light is detected and before the light source is automatically powered off.

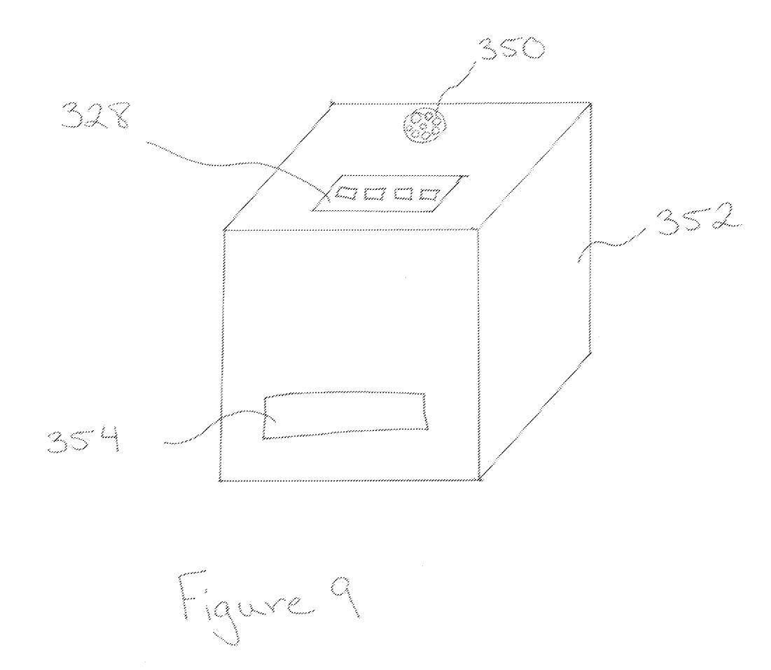

FIG. 8 depicts a perspective view of the light source sensor 320, such as may be used to detect the light 360 emitted from one or more exit points 318. The sensor 320 may be portable and may include a power source 331, such as a battery, a power cord plugged into an electrical receptacle, through connection with the light source 322 or other source or electrical power. The sensor 320 may be powered on or off by depressing a power button or switch 335. The sensor 320 may be capable of detecting the wavelength(s) of light 360 generated by the light source 322. For example, the sensor 320 may be a complementary-metal-oxide semiconductor (CMOS) image sensor or a charge-coupled device (CCD) image sensor. The sensor 320 may include one or more photodiodes, photovoltaic cells, or other light detecting materials. The sensor 320 may be handheld, such as to allow a user to move the sensor 320 around a person's body to detect transmitted light 360. The sensor 320 may be large enough such that the sensor 320 may detect the light 360 transmitted through an exit point/s 318 in the desired region of the person without moving the sensor 320. The sensor 320 may include a scanner 324 which may identify and store barcode or QR code identification through CCD or laser, among others. The scanner 324 may be used to identify and. store information. The scanner 324 may be used external to a person's body and may be used to scan a wristband, identification card or other materials. In one or more embodiments the scanner 324 may track and store times and/or dates the system is used to check the location of the feeding tube 314.

The sensor 320 may detect light 360 transmitted through an exit point 318 at or some distance away from the person, or some distance away from the skin of the person. In one or more embodiments, the sensor 320 may measure the intensity of light 300 being detected. For example, the sensor 320 may be moved about an abdominal area of the person and the sensor 320 may detect a faint presence of light 360. As she sensor 320 is moved closer to the exit point/s 318 on the distal end 316 of the feeding tube 314 the sensor 320 may detect a greater intensity of light 360. The sensor 320 may detect the location on the person's body through which a threshold intensity of light 360 is transmitted (e.g., the “hot spot”). The “hot spot” may indicate the location of the exit point/s 318, such as at or near the distal end 310 of the feeding tube 314 within the person.

In one or more embodiments, the sensor 320 may defect and identify the different wavelengths of light 360 transmitted by the light transmitting element 346. The sensor 320 may identify the different wavelengths of light 360 and determine the rotational orientation of the tube 314 based on the detection and identification of the wavelength of light 360. In one or more embodiments, the sensor 320 may detect and identify the different frequency and magnitude of oscillations of the light 360. The sensor 320 may be able to determine the rotational orientation of the tube 314 based on the frequency and magnitude of oscillations of the light 360.

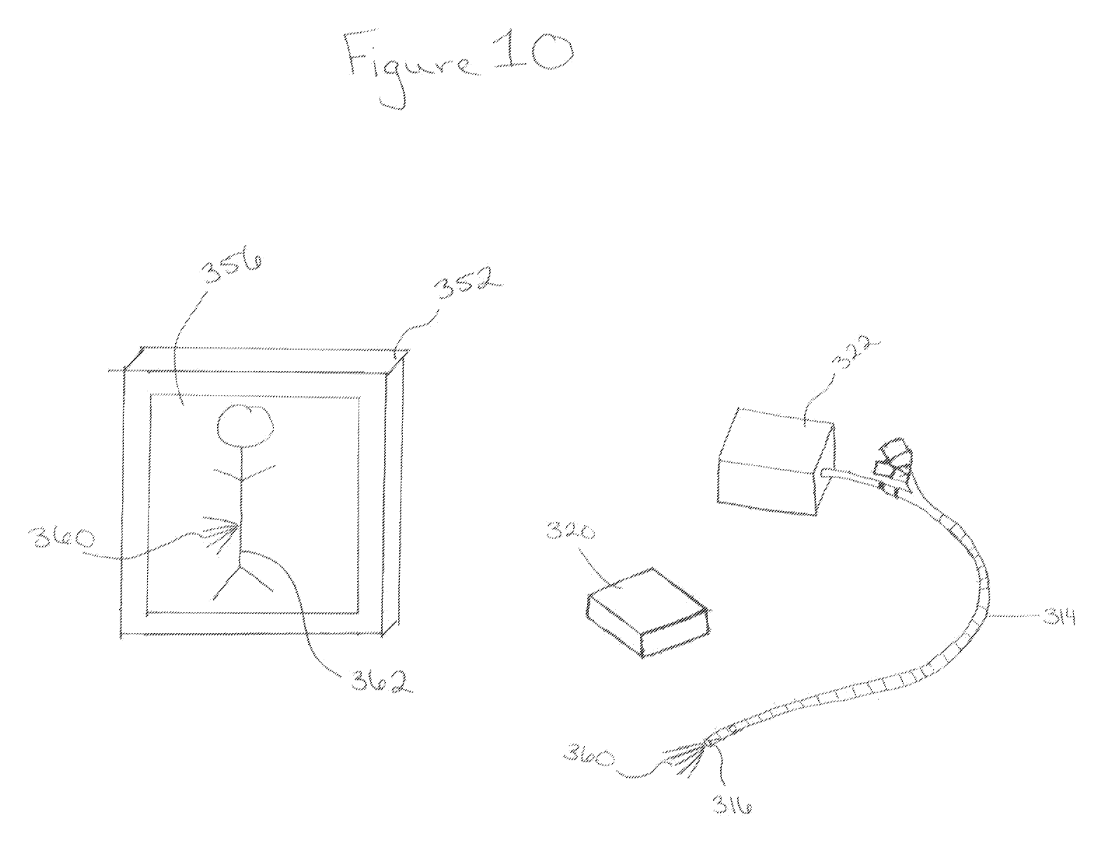

The sensor 320 may generate the indicator signal or the sensor 320 may communicate wired or wirelessly to a receiver 352, as shown in FIG. 9, which may include a signal generator 354. The sensor 320 or receiver 332 may include a speaker 350 that may produce an audible signal at or around the time the sensor 320 detects the light 360. The sensor 320 or receiver 352 may include one or more Light Emitting Diodes (LEDs) 328, which may produce a visual signal indicating that the sensor 320 has detected the light 360. In one or more embodiments, both an audible and a visual signal may be generated. In one or more embodiments, the signal generated may change as a function of the distance between the sensor 320 and the “hot spot”. For example, the change may be one of tone, pitch, frequency or the like when the signal is audible. Alternatively or in addition, the sensor 320 or receiver 352 may include one or more lights that flash, the frequency of the flash may increase the closer the sensor gets to the exit spot 318, such as the exit spot 318 on the distal end 316 of the tube 314. The sensor 320 or receiver 352 may include a series of LEDs 328 on the sensor 320 or receiver 352 and the LEDs 328 may display a series of signals indicating the proximity of the sensor 320 to the “hot spot”.

As depicted in FIG. 10, in one or more embodiments, the sensor 320 or receiver 352 may include a user interface 356. The user interface 356, may depict an image of the person 362 with the location through which the light 360 emitted from the distal end 316 of the feeding tube 314 superimposed over the image of the person 362. In one or more embodiments, the receiver 352 may be a user interface 356 that may depict an image of a portion of the person. The image may also depict an image of an area, other than the entire body of the person, where the sensor 320 is expected to detect light 360 if the feeding tube 314 is properly placed. The images may be stored in the sensor 320 or receiver 352 for a user to open or download at a later time.

In one or more embodiments, markings, such as temporary tattoos or reflective stickers, may be placed on locations, such as important or landmark locations, on the person's body. The markings may be reflective or contain an active light source to allow the sensor 320 to detect the markings. In one or more embodiments the sensor 320 may include one or more LEDs directed toward the markings, to allow the reflective markings to be detected. These markings may demarcate a zone in which the light 360 may be detected if the feeding tube 314 is properly placed. Generally, if the sensor 320 or receiver 352 indicates teat the light 360 has been detected within the zone indicated by the markings the feeding tube 314 is properly placed. A location indicator may be made on the person to indicate detection of a “hot spot”, which may allow a user to determine if a feeding tube 314 has shifted or moved the next time the system is used.

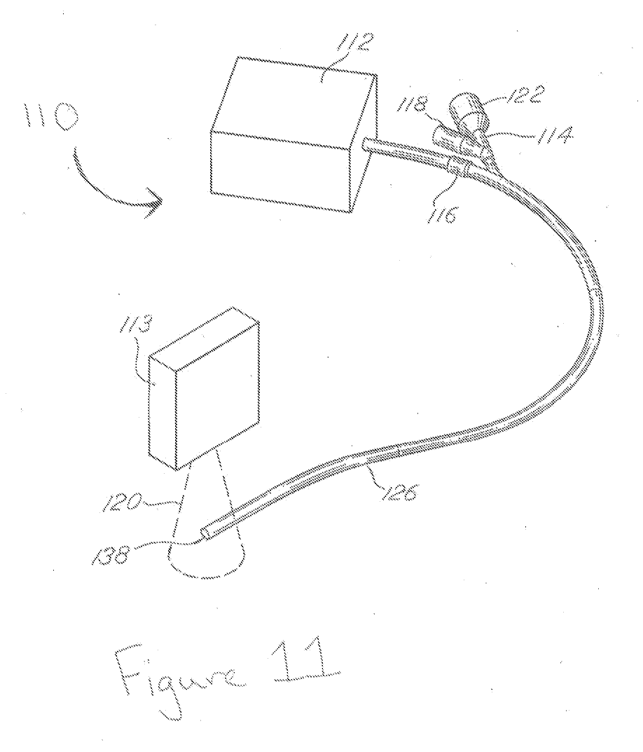

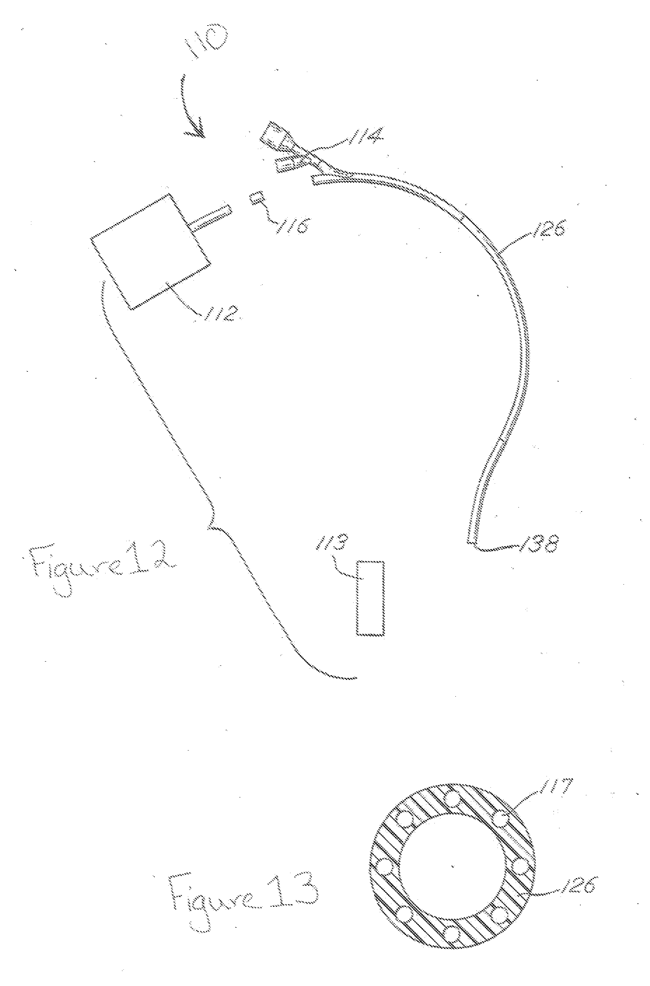

FIGS. 11 and 12 illustrate a perspective and blown up view of an example of a feeding tube system 110, respectively. The feeding tube 110 has a “Y” port 114 to administer feedings, medications or other treatments. The “Y” port 114 may comprise dual ports 118 and 122 such that multiple feedings, medications or other treatments may be administered simultaneously. For example, the port 118 may be used to receive gastric feeding and the port 122 may be used for medication, flushing, or as a racking port, or vice versa. The “Y” port 114 is connected to a main tube 126. The main tube 126 administers feedings, medications or other treatments to a person internally, for example, through the stomach or the small intestine. The tube 126 may also be referred to as a “feeding tube,” an “oral gastric tube (or OG-tube),” a “nasogastric tube (or NG-tube),” or an “intestinal tube,” as it refers to the location of the tube when inserted into the person. The tube 126 comprises a distal end 138, which may comprise a hole or holes for the administration of fluid into the person. The feeding tube 126 comprises a light source sensor 117, which is connected to a receiver 112 via a joint 116. The joint 116 provides for removable connection of the tube 126 to the receiver 112. For example, when not in operation, the tube 126 may be disconnected to the receiver 112. In one or more embodiments, the light source sensor 117 may be wireiessiy connected to the receiver 112.

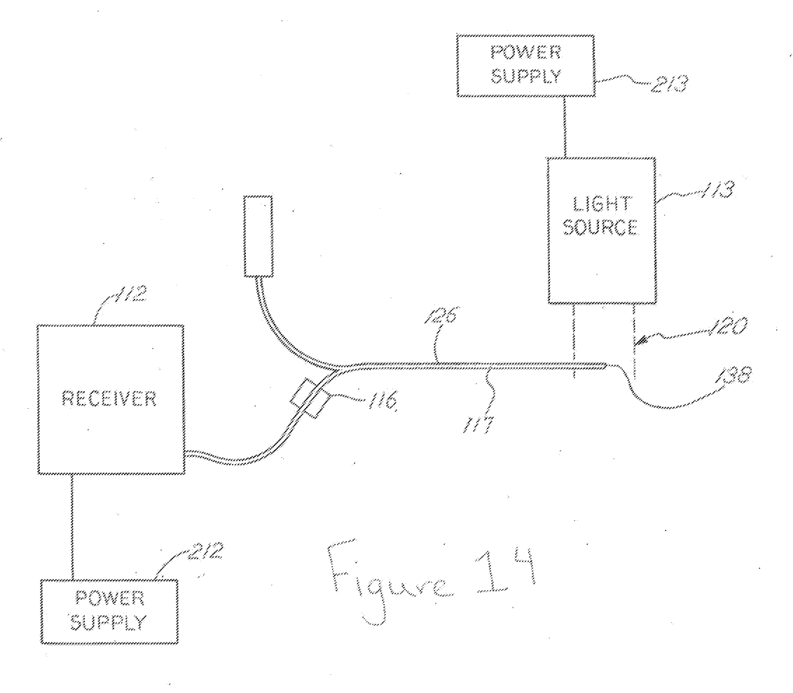

A cross section of the tube 126 is depicted in FIG. 13. The feeding tube 126 may foe comprised of a variety of flexible materials suitable for insertion into a person. A material such as medical grade polyurethane is, by way of example only, a suitable material type. In one or more embodiments the feeding tube may comprise a radiographic pigment for X-ray detection. For example, the tube 126 may have a radiographic pigment stripe that will appear on an X-ray, indicating the position of the tube in the body. In one or more embodiments the feeding tube 126 may comprise a series of markings, similar to the markings 30 of the tube 26 in FIG. 1. The markings may be used as a guide to determine the approximate location of the feeding tube inside the person by indicating the amount of tube exposed. For example, the markings may foe numbered and consistently spaced apart to measure length in, by way of example only, centimeters. Thus, a practitioner may insert the tube 126 to an approximately appropriate depth before attempting to locate the tube in the person.

A light source sensor 117 runs along the outer wall of the tube 126 as shown in FIG. 11. The light source sensor may be, for example, a passive infrared sensor or a fiber-optic filament. In one or more embodiments the light source sensor 117 is a plurality of fiber-optic filaments, such as is depicted in FIG. 11. The end of the light source sensor 117 is situated at the distal end 138 of the feeding tube 126. In one or more embodiments, a feeding tube 126 may have more than one light source sensor; however, one or more embodiments will employ only one light source sensor.

A light source 113 shines a non-radiographic radiation such as light 120, that does not show up on an x-ray film, for example, through the body as shown in FIG. 11. In one or more embodiments, the light source is an infrared light source, and the light shone is infrared light. When the light 120 is detected by the end of the light source sensor 117, a detection signal is transmitted up the sensor 117 to the receiver 112. In one or more embodiment, the light source sensor 117 may appreciate a range of light intensity as opposed to merely the presence of light, or lack thereof. For example, the light source sensor 117 may detect the faint presence of the light source 113 when the light is within a particular range of the sensor 117. As the light source 113 moves closer to the distal end 138 of the tube 126 the intensity of light at the sensor 117 increases, and as the source 113 moves away from the end 138 of the tube, the light intensity at the sensor 117 decreases. In one or more embodiments, the light source sensor 117 appreciates this change, and the receiver 112 generates an indicator signal to indicate that the light source 113 is closer to or farther from the end of the light source sensor 117 accordingly.

FIG. 14 depicts a logical block diagram of an embodiment employing the use of a light source sensor to locate a feeding tube inside of a person in operation. A light source 113 is connected to a power supply 213. Similarly, receiver 112 is connected to a power supply 212. Power supplies 212 and 213 may be, for example, a 12-volt power supply, a battery, an alternating current source or an alternate power source. An operator moves the light source 113 externally above a person in an area where the distal end 138 of the feeding tube 126 is approximately located. For example, when the feeding tube is intended to be placed inside of a person's stomach, an operator may move the light source 113 externally, approximately above the person's stomach.

When the tight source is within the detected range of the light source sensor 117 embedded in the tube 126, the receiver 112, connected to the light source sensor 117, generates an indicator signal indicating that the light 120 has been perceived by the light source sensor 117. For example, the receiver may emit a sound indicating the light is detected, or may cause a sound to be emitted from another source such as a speaker. Alternatively, the receiver 112 may indicate perception of the light 120 through visual signals, such as an LED light or lights that blink, change color or otherwise indicate the light signal is perceived. The receiver 112 may also cause a display unit to produce a signal, or a message that indicates that the light source sensor 117 detects the presence of light, or lack thereof.

In one or more embodiments, as the light 120 draws nearer the actual location of the light source sensor 117, the intensity of the light detected increases. Accordingly, the intensity of the signal generated by the receiver 112 may increase. The receiver 112 may indicate a perceived increase in intensity by changing or modifying the signal produced. For example, the receiver 112 may produce a sound, such as a tone, that increases in pitch or volume as the intensity of light perceived increases. In one or more embodiments the receiver 112 may produce a series of intermittent sounds wherein the amount of time between the sounds increases or decreases with an increase in intensity of light perceived by the light source sensor 117. For example, where the light source sensor 117 detects no light, the receiver 112 may produce a “chirp” once every two seconds, or not at all. As the light source sensor 117 detects an increase in the intensity of light, the receiver may reduce the time between the “chirps” produced, such that when the light intensity perceived by the light source sensor 117 is at a maximum, the receiver produces one “chirp” every 0.1 seconds, for example.

Alternatively or additionally, the receiver 112 may have a visual display to generate the signal. For example, in one or more embodiments, the receiver 112 may have a blinking light that modifies the frequency of the blinking with a change in intensity. Additionally, the receiver may provide a series of LED lights, where the particular light, or the number of lights illuminated, indicates the intensity of the signal. For example, the receiver may have a bar of ten lights on the receiver, each light corresponding to a particular intensity level. When the intensity of light detected at the light source sensor 117 is at a maximum, the light or lights corresponding to maximum light detection is illuminated, for example. In one or more embodiments, the receiver 112 may provide a quantitative value of the intensity of the signal. For example, the receiver 112 may be connected to a monitor or a display module that produces a numerical or quantitative value indicating the intensity of light perceived by the right source sensor 117. In one or more embodiments, the receiver may generate both an audible and a visual indicator signal.

The operator may therefore determine an accurate position of a feeding tube inside of a person by locating the light source position that yields the highest intensity indicator signal, as produced by the receiver. In one or more embodiments, a predetermined intensity value may confirm the presence of the light source sensor 117 and thus the feeding tube 126. For example, where the receiver 112 has a light display ranging in intensity level from zero to ten, wherein a value of zero indicates no perceived light, and 10 indicates a maximal or near maximal amount of perceived light, it may be predetermined that an intensity level of 7 or greater is sufficient to confirm the location of the tube 126.

In addition to the systems described, techniques for locating the position of a feeding tube inside of a person's body are also provided. In one or more embodiments, the technique comprises the following steps:

1. Providing a feeding tube having a distal end with a proximity target. The proximity target may be a metal band as disclosed above.
2. Inserting the feeding tube into the person. In one or more embodiments, the tube may be inserted through the nose or the mouth. In one or more embodiments the tube may pass through the esophagus into the stomach. In one or more embodiments, the tube may pass beyond the stomach and into the small intestine.
3. Placing a detector above the person. For example, where a feeding tube having a metal band is placed inside of a person's stomach, a metal detector may be placed above the person's stomach area.
4. Generating a signal, indicating the detection of the proximity target. For example, where the detector is a metal detector, the detector may produce a sound indicating the detected presence of the target. In one or more embodiments, the detector may produce a visual signal in addition to, or instead of, an audible signal indicating the detected presence of the target.

A technique fox locating the position of a feeding tube 314 inside a person, may include a sensor 320 such as a light source sensor external to the body of the person. The sensor 320 may be used for detecting light 360 transmitted through an exit point 313 at or near the distal end 310 of the feeding tube 314 inside the person. In one or more embodiments the technique may include:

1. Providing a feeding tube 314 and a light transmitting element 346 configured to transmit light 360 through an exit point 318 near the distal end 316 of the feeding tube 314. The light transmitting element 340 may be embedded in or coupled to the feeding tube 314. The light transmitting element may be inserted into a lumen 340 or 342 of the feeding tube 314 prior to or after placement and may be removable or permanent.
2. Inserting the feeding tube 314 and the light transmitting element 346 into a person such that the distal end 316 is located within the person. For example, the feeding tube 314 may be inserted through the nose or mouth of a person, through the esophagus and into the stomach or small intestine. In some embodiments, the feeding tube 314 may be inserted into the person before the insertion of the light transmitting element 346 into the feeding tube 314. In other embodiments, the feeding tube 314 and light transmitting element 346 may be inserted simultaneously.
3. The light transmitting element 346 may be coupled to a light source 322 sad light 360 sues be transmitted from the light source 322, along the light transmitting element 346 to a distal end of the light transmitting element 346, and through an exit point 318 at or near the distal end 316 of the feeding tube 314. For example, a ferrule based connector may be used to couple an infrared light source 322 to fiber optic filaments embedded in a lumen 340 or 342 of the feeding tube 314 and the filaments may extend from the light source 322 to one or more exit points 318 of the tube 314.
4. A light source sensor 320 may be external to the person in the general region the user mould expect the distal end 316 of the feeding tube 314 to be located. For example, a user may sieve the light source sensor 320 around the abdomen or chest area of the person.
5. A signal may be generated at or around the time the sensor 320 detects the light 360 transmitted through an exit point 318 at or near the distal end 316 of the feeding tube 314. The signal may be generated based on the intensity of light 360 detected. The signal may be generated by the sensor 320 or by a receiver 352 coupled to the sensor 320. For example, the sensor 320 or receiver 352 may generate a sound that increases in pitch, volume, or frequency as the intensity of the light increases. Alternatively or additionally, the sensor 320 or receiver 352 may include a visual signal. By way of example, the visual signal may be generated by a light emitting diode (LED) 328 which may turn on, blink (e.g., flash), or vary in color based on the presence, intensity, wavelength or frequency of light 360 the sensor 320 detects. In one or more embodiments the sensor 320 may be coupled to the light source 322 such that at or around the time the light 360 is detected the sensor 320 powers off the light source 322.

The above listed technique may include any or all of the listed steps and the order of the steps may vary. Accordingly, the numbering of the above steps or operations is merely for convenience of discussion and should not be limiting in any way.

Other embodiments provide techniques for locating the position of a feeding tube inside of a person, where the sensor is a light source sensor in the feeding tube inserted into the person, detecting a light source external to the body of the person. The technique includes:

1. Providing a feeding tube having a light source sensor at the distal end.
2. In one or more embodiments, the light source sensor of the feeding tube is connected to a receiver. The receiver or sensor may be located exterior to the person's body.
3. In one or snore embodiments, the feeding tube having the light source sensor is then inserted inside of a person. For example, the feeding tube may pass through a person's nose or mouth, down the esophagus into the stomach of the person, or beyond the stomach and into the small intestine. In one or more embodiments, the feeding tube may be inserted into the person before the light source sensor is connected to the receiver. For example, the operator or technician may prefer not to have the sensor connected to a receiver while inserting into the person in order to have a greater available range of movement of the tube.
4. A non-radiographic radiation such as light is shone externally above the person. For example, a user may shine an infrared light from a light source above an area of the person's body approximately where the feeding tube is expected to be located.
5. An indication signal is generated reflecting the detection of light by the light source sensor. In one or more embodiments, the signal generated may be based upon the intensity of the light perceived. For example, the receiver may generate a sound that increases in pitch or volume as the intensity of the light detection signal transmitted by the light source sensor increases. Alternatively, the receiver may produce an intermittent sound that increases in frequency with an increase in intensity based on the intensity of the light perceived. In one or more embodiments the indication signal may be visual. For example, in one or more embodiments a light source may foe provided that varies in color, blinking frequency, or amount of lights powered based on the intensity of the light detection signal received by the receiver or sensor.
6. In one or more embodiments, the position of the feeding tube within the person's body is located by moving the right source over the body to the point where the indication signal is of highest intensity. For example, an operator may move the light source about a person's stomach and note the location that causes the receiver to generate the signal indicating the highest perceived intensity of light. In one or more embodiments, the light source may be moved to a location where the indicator signal reaches or exceeds an intensity level that is predetermined to confirm the presence of the light source sensor, and thus the feeding tube.

The above mentioned steps are not limited to be performed in the order in which they are listed. For example, the feeding tube may be inserted into a person first, after which the light source sensor in the tubing may be connected to a receiver.

The different embodiments of the presently described techniques and systems offer techniques that provide several advantages over conventional techniques of detecting the position of a feeding tube inside of a person. The presently described techniques are more accurate than those provided by use of pH, gastric enzymes, air or CO2 detectors. Furthermore, the techniques allow for the location of the tube to be easily confirmed at any time during use of the feeding tube, not just during insertion. Additionally, the present techniques do not rely on radiographic techniques to detect the position of the feeding tube. Furthermore, the detector is simple and easy to use by either medical professionals or medically trained friends and family of the person, and thus the techniques may be used either at a medical facility or a home care setting.

Variations and modifications of the foregoing are within the scope of the present technology. It is understood that the technology disclosed and defined herein extends to all alternative combinations of two or more of the individual features mentioned or evident from the text and/or drawings. All of these different combinations constitute various alternative aspects of the present technology. The embodiments described herein explain the best modes known for practicing the technology and will enable others skilled in the art to utilize the technology. The claims are to be construed to include alternative embodiments to the extent permitted by the prior art.

What is claimed is:

1. A system For detecting the location of a distal end of a feeding tube in a person's body, comprising;
a feeding tube comprising a distal end and a proximal end, the feeding tube configured tor insertion into the person;
a light source configured to generate light;
a light transmitting element arranged within the feeding tube, extending from the light source to an exit point of the feeding tube and configured to be coupled to the light source and transmit the light from at or near the proximal end of the feeding tube and through the exit point at or near the distal end of the feeding tube;
a light source sensor configured to detect the light; and a signal generator in communication with the light source sensor configured to generate an indicator signal at or around the time the light source sensor detects the light transmitted through the exit point near the distal end of the feeding tube.

2. The system of claim 1, wherein the light source sensor is coupled to a receiver.

3. The system of claim 2, wherein the receiver further comprises a user interface configured to display an image.

4. The system of claim 2, wherein the receiver includes the signal generator.

5. The system of claim 1, wherein the light transmitting element comprises at least one fiber optic filament embedded in a lumen of an outer wall of the feeding tube.

6. The system of claim 1, wherein the light transmitting element comprises a stylet configured to transmit light.

7. The system of claim 1, wherein the light transmitting element comprises optically transmittable materials.

8. The system of claim 1, further comprising multiple exit points along a length of the feeding tube through which the light can be transmitted.

9. The system of claim 1, wherein the light is one of visible and infrared light.

10. The system of claim 1, wherein the light source is configured to oscillate the intensity of the light.

11. The system of claim 1, wherein the signal generated is audible.

12. The system of claim 1, wherein the signal produced is visual.

13. The system of claim 1, wherein the signal generated is varied based on the intensity of light detected by the light source sensor.

14. The system of claim 1, wherein the light source sensor is configured to be held.

15. A method tor detecting the location of a feeding tube in a person comprising:
inserting a feeding tube into a person such that a distal end of the feeding tube is located within the person;
generating light such that the light is transmitted from a proximal end of the feeding tube to an exit point at or near the distal end of the feeding tube;
detecting light transmitted from the exit point; and
generating an indicator signal as a function of the detected light.

16. The method of claim 15, wherein generating light from a proximal end of the feeding to an exit point at or near the distal end of the feeding tube includes transmitting the light using a light transmitting element wherein the light transmitting element is uncoupled from the feeding tube after use.

17. The method of claim 15, wherein detecting the light transmitted from the exit point includes detecting the light using a light source sensor, and wherein generating the indicator signal includes generating the indicator signal using a receiver coupled to a light source sensor.

18. The method of claim 15, further comprising oscillating the intensity of the light generated.

19. The method of claim 15, comprising generating a sound in response to receiving the indicator signal; and varying a pitch or volume of the sound as a function of the indicator signal.

20. A system for detecting the location of an exit point of an object in a patient's body, comprising;
a feeding tube comprising a distal end and a proximal end, the feeding tube configured for insertion into the patient;
a light source configured to generate and oscillate an intensity of one of visible and infrared light;
at least one fiber optic filament or stylet embedded in a lumen of the feeding tube configured to be coupled to the light source and transmit the light from at or near the proximal end of the feeding tube and through a plurality of exit points distributed along the length of the feeding tube including an exit point at or near the distal end of the feeding tube;
a handheld light source sensor configured to detect the light; and
a receiver coupled to the light source sensor, the receiver comprising:
a signal generator configured to generate an audio or visual indicator signal at or around the time the light source sensor detects the light transmitted through the exit point near the distal end of the feeding tube, the signal generated is varied based on the intensity of light detected, by the light source sensor.
